(19) United States
(12) Patent Application Publication
(10) Pub. No.: US 2026/0080618 A1
(43) Pub. Date: Mar. 19, 2026

(54) DETERMINING THE POSITION OF AN IMPLANT IN A HOLLOW ORGAN

(71) Applicant: Siemens Healthineers AG, Forchheim (DE)

(72) Inventors: Markus Kowarschik, Erlangen (DE); Andreas Berting, Schlüchtern (DE)

(21) Appl. No.: 19/327,422

(22) Filed: Sep. 12, 2025

(30) Foreign Application Priority Data
Sep. 13, 2024 (DE) 10 2024 208 714.9

(51) Int. Cl. G06T 17/00; A61B 34/10; G06T 7/11; G06T 7/33; G06T 7/73

(52) U.S. Cl. CPC G06T 17/00; A61B 34/10; G06T 7/11; G06T 7/337; G06T 7/73; A61B 2034/102; G06T 2207/10016; G06T 2207/20216; G06T 2207/30052; G06T 2207/30101; G06T 2207/30204; G06T 2210/41

(57) ABSTRACT

In order to determine the position of an implant in a hollow organ, image data is obtained consisting of one or more two-dimensional images, that each represent the implant in a first state inside the hollow organ from at most ten different viewing directions. A three-dimensional first reconstruction of the implant in the first state is generated on the basis of the image data. The image data includes two to ten two-dimensional images having different viewing directions with respect to the implant. In addition, the image data includes two or more two-dimensional first images, each of which represents the implant in the first state according to a first viewing direction at different points in time. At least one averaged first image is generated on the basis of the two or more first images, and the first reconstruction is made on the basis of the at least one averaged first image. A representation of the first reconstruction is displayed on a display device.

### CROSS REFERENCE TO RELATED APPLICATIONS

This application claims the benefit of DE 10 2024 208 714.9 filed on Sep. 13, 2024, which is hereby incorporated by reference in its entirety.

### FIELD

Embodiments relate to a computer-implemented method for determining the position of an implant in a hollow organ, where position shall be understood to mean the spatial position, location, orientation and/or shape, and to a data processing system for performing the computer-implemented method, to an imaging system having the data processing system, and to associated computer program products.

### BACKGROUND

For many patients with cerebral aneurysms, the implantation of a stent for flow diversion, also known as a flow diverter, flow diverting stent or flow diverting implant, constitutes the treatment method of choice. In the case of an endovascular intervention, such an implant is inserted in the artery in which the aneurysm bulge arises in order to recanalize the blood stream and to reduce the blood flow into the aneurysm itself, so that thrombosis occurs in the aneurysm and the aneurysm eventually degenerates. The position of the implant in the blood vessel is crucial to the success of the treatment. Angiography systems, for example X-ray angiography systems, for example, may be used to determine the position of the implant. Biplane X-ray angiography systems may also be employed, for example, in order to determine the position of the implant and to provide the person performing the treatment with two views simultaneously from different viewing directions. This is also referred to as biplane X-ray fluoroscopy.

Stents for flow diversion are available in various sizes, for example with different lengths and diameters. Both the choice of the suitable implant and its usage may present a challenge and require a high degree of experience. Usually, the choice of the implant and planning the correct position of the implant in the artery are based on a pre-intervention 3D angiographic image, a CTA image or a cone-beam CTA image (CTA: computed tomography angiography).

It is generally desirable to use an angiography system to determine the position of the implant in the blood vessel, because usually the insertion of the implant is also assisted by an angiography system, and therefore the position may advantageously be determined immediately using the same angiography system without a change in device. Furthermore, it is usually desirable to determine the position not by a cone-beam CTA image, because this is relatively time consuming, but by a limited number of images generated by the angiography system from a limited number of viewing directions.

When using single-plane angiography systems to determine the position, it is very difficult for the person performing the treatment to judge the three-dimensional location and/or shape of the implant. Although two live images are available in the case of biplane X-ray fluoroscopy, again a limiting factor here is the experience of the person performing the treatment in judging the three-dimensional location and/or shape of the implant from two two-dimensional images. In the case of stents for flow diversion, there is the added complication that this type of stent is usually arranged initially in a catheter, that is then removed piece by piece and releases the stent, that accordingly deploys dynamically until it reaches its final location and shape.

Similar problems also arise with other implants applied by endovascular procedures, for example with what are known as aneurysm coils, intra-aneurysmal flow diverters or other neuro stents for treating intracranial stenosis of vascular segments. In addition, similar situations may also arise in usages outside the brain, for example when applying renal artery stents, stents in bile ducts, and so on. Finally, they may also arise when applying implants in other hollow organs. This approach is not necessarily limited to the use of endovascular implants. The use of implants in other hollow organs may involve similar challenges.

A full three-dimensional determination of the position of the implant using a CT system would allow a reliable assessment of the location and/or shape of the implant, but is usually not feasible because of the large amount of effort and the associated X-ray dose.

The publication J. C. H. Schuurbiers et al.: “In Vivo Validation of CAAS QCA-3D Coronary Reconstruction Using Fusion of Angiography and Intravascular Ultrasound (ANGUS)”, Catheterization and Cardiovascular Interventions 73:620-626 (2009) describes how a three-dimensional approximate reconstruction of blood vessels may be generated on the basis of just two two-dimensional images having different viewing directions.

### BRIEF SUMMARY AND DESCRIPTION

The scope of the embodiments is defined solely by the appended claims and is not affected to any degree by the statements within this summary. The present embodiments may obviate one or more of the drawbacks or limitations in the related art. Independent of the grammatical term usage, individuals with male, female or other gender identities are included within the term.

Embodiments provide visual assistance to a person performing a treatment in determining the position of an implant in a hollow organ, which assistance offers greater reliability and manages without full three-dimensional imaging.

Position refers to the spatial position, location, orientation and/or shape of the implant in the hollow organ. Thus good visibility and identifiability of the implant is required in order to determine the position. A sufficiently high image quality in the representation of the implant is needed for the implant to be visible and identifiable. Embodiments improve the quality of the representation of an implant in a hollow organ without full three-dimensional imaging.

Embodiments are based on the concept of generating a three-dimensional reconstruction of the implant on the basis of two-dimensional images according to a limited number of at most ten different viewing directions and displaying a representation of the reconstruction on a display device. The number of viewing directions is two to ten, and/or the reconstruction is additionally generated on the basis of a geometric and/or mechanical model of the implant.

According to an embodiment, a computer-implemented method is defined for determining the position of an implant in a hollow organ. In this method, image data is obtained consisting of one or more two-dimensional images, which images each represent the implant in a first state inside the hollow organ from a total of at most ten different viewing directions. A three-dimensional first reconstruction of the implant in the first state is generated on the basis of the image data. The image data includes two to ten two-dimensional images having different viewing directions with respect to the implant, and/or the first reconstruction is generated on the basis of the image data and a predefined geometric and/or mechanical model of the implant. A representation of the first reconstruction is displayed on a display device.

According to an embodiment, the image data includes two or more two-dimensional first images, each of which represents the implant in the first state according to a first viewing direction at different points in time. An averaged first image is generated on the basis of the two or more first images. The first reconstruction is generated on the basis of the averaged first image.

Each of the first images thus corresponds to the same viewing direction, namely the first viewing direction. This does not rule out the possibility that the image data includes further images corresponding to other viewing directions.

By the averaging of the two or more first images, noise in the two or more first images may be suppressed effectively, while the outline of the implant as depicted in the two or more first images is retained or is effectively enhanced or represented more clearly as a result. In other words, the signal-to-noise ratio is increased. Consequently, for generating the first reconstruction, the shape and/or location of the implant in the particular averaged image may be ascertained or extracted more accurately and more reliably, leading to more accurate and reliable generation of the first reconstruction.

Unless stated otherwise, all the steps of the computer-implemented method may be performed by a data processing system that includes at least one data processing device. For example, the at least one data processing device is configured or adapted to execute the steps of the computer-implemented method. For this purpose, the at least one data processing device may store, for example, a computer program containing commands that, when they are executed by the at least one data processing device, cause the at least one data processing device to execute the computer-implemented method. The computer-implemented method may also be implemented entirely or partially in hardware. The expressions “data processing system” and “at least one data processing device” may be used interchangeably here and below. This also applies to corresponding expressions derived therefrom.

For the case in which the at least one data processing device includes two or more data processing devices, certain steps implemented by the at least one data processing device may also be understood to mean that different data processing devices implement different steps or different parts of a step. For example, it is not necessary for each data processing device to implement the steps. In other words, the implementation of the steps may be distributed over the two or more data processing devices.

From each embodiment of the computer-implemented method is obtained a corresponding embodiment of a method for determining the position of an implant in a hollow organ, which method is not purely computer-implemented and is obtained by incorporating corresponding steps for generating the image data.

The determining of the position of the implant in the hollow organ is realized by the single or, if applicable, multiple or repeated or continuous generation of the first reconstruction as described and the representation of each first reconstruction on the display device. The position of the implant in the hollow organ is thereby determined for example also repeatedly for different successive points in time.

The implant may differ in terms of its position, location and/or orientation in the three-dimensional space and/or in terms of its geometric shape at different successive points in time. For example in the case of flow diverting implants for taking blood flow in the hollow organ past an aneurysm, or other implants that are inserted into blood vessels of the brain, the implant may be introduced initially into the hollow organ in a compressed state, for example enclosed by a vascular catheter, and may be gradually both navigated to an intended position and/or location and deployed, for example by withdrawing the vascular catheter so that the implant may assume its final shape.

The state of the implant may thus refer, for example, to its current location and/or geometric shape in the three-dimensional space. The fact that the two-dimensional images in the image data each represent the implant in the first state may therefore be understood to mean that the location and/or shape of the implant in the two-dimensional images in the image data is identical or substantially identical. Since the deployment of the implant takes place inside the hollow organ typically in a timescale that is significantly greater than the time taken to acquire a two-dimensional image, for instance an X-ray image, it may also be assumed to be a good approximation when acquiring a multiplicity of images within a timespan that is short compared with the time taken for the implant to deploy, that the implant is in a stationary or quasi-stationary state in the images in the image data.

The images in the image data represent the implant from at most ten different viewing directions, so from a number of viewing directions that lies in the range of one to ten. It may be the case here that the image data consists of one to ten two-dimensional images, with each of the images corresponding to another viewing direction. In the limiting case of a single viewing direction, it could also be the case that the image data consists of one two-dimensional image having just this viewing direction. It is also possible, however, that for the number of one to ten viewing directions, the image data may contain two or more two-dimensional images for each viewing direction.

For example, the three-dimensional first reconstruction may be generated on the basis of the image data without using further image data representing the implant at another, subsequent point in time. This does not rule out the possibility that, for example, pre-intervention image data is used, for example used additionally, in order to generate the first reconstruction. The procedure in accordance with the computer-implemented method differs from full three-dimensional monitoring, for example by a CT system or a CBCT system, in that the number of different viewing directions is drastically reduced compared with the last-mentioned techniques.

Thus the first reconstruction may be understood as an approximate three-dimensional reconstruction of the implant. An exact or definitive reconstruction may require depictions from a far higher number of viewing directions, for instance in accordance with the principles of computed tomography. The present case, however, may exploit the fact that the implant cannot assume any shape in the three-dimensional space. Instead, the final shape or approximate final shape, as well as the initial shape or approximate initial shape of the implant, is known. For states between the initial shape and the final shape, suitable interpolation may be performed or such states may be known. A three-dimensional approximate reconstruction of this type is also referred to as a symbolic reconstruction of the implant.

Thus, in contrast with traditional CT methods or CBCT methods, the solution space is drastically restricted for the generation of the first reconstruction. For example, the known shape of the implant, or certain aspects of the known shape, for instance symmetry properties, length, diameter, and so on, may be used in the generation of the first reconstruction to restrict the solution space. This is why it is possible to generate a good approximation of the three-dimensional shape in the form of the first three-dimensional reconstruction of the implant even using a small number of viewing directions, and in the extreme case using just a single viewing direction. If there is just a single viewing direction, however, it is necessary to use a geometric and/or mechanical model of the implant, that specifies, for example, geometric properties such as the shape of the implant, symmetry properties of the implant, a diameter or length, or the like.

The geometric and/or mechanical model may also include symmetry properties, for example rotational symmetry properties, of the implant, or the like. If two or more viewing directions are available, a geometric and/or mechanical model of the implant is not absolutely necessary, but it may increase the accuracy of the first reconstruction in some embodiments.

In a variant of the computer-implemented method, the image data includes two to ten two-dimensional images having different viewing directions with respect to the implant, and the first reconstruction is generated on the basis of the image data and independently of a geometric and/or mechanical model of the implant. In another variant, the image data includes two to ten two-dimensional images having different viewing directions with respect to the implant, and the first reconstruction is generated on the basis of the image data and on the basis of the predefined geometric and/or mechanical model of the implant. According to a further variant, all the images in the image data correspond to the same viewing direction, and the first reconstruction is generated on the basis of the image data and the predefined geometric and/or mechanical model.

If the image data includes the two to ten two-dimensional images with different viewing directions, then this may be interpreted to mean that the viewing direction for all the images of the two to ten two-dimensional images is different in each case. In addition, however, it is also possible that the image data includes more than just one two-dimensional image per viewing direction.

In order to generate the first reconstruction, a method may be used as described, for example, in the publication by Schuurbiers et al. cited above. It should also be pointed out, however, that in the cited publication, a suitable reconstruction is generated for the blood vessels themselves and not for an implant in a blood vessel or the like. In the present case that as a result of the defined shape of the implant, such an algorithm for the symbolic reconstruction of blood vessels may also be used for the symbolic reconstruction of the implant.

The two-dimensional images may be images from different sources, depending on the embodiment of the method. In general, they are images that were generated by a medical imaging modality. For example, they may be X-ray images, for example two-dimensional X-ray projection images. Similarly, however, two-dimensional images from magnetic resonance tomography systems or other imaging modalities may also be used.

The computer-implemented method provides the person performing the treatment with the representation of the first three-dimensional reconstruction, so that the person performing the treatment may assess the first state of the implant, and also assess further states of the implant dynamically when the method steps are accordingly carried out repeatedly. For example, a direct three-dimensional representation of the implant is thereby provided, without the person performing the treatment having to make a more or less reliable interpretation of the implant from two or more simultaneously displayed two-dimensional images in order to be able to deduce the current shape and/or location of the implant. Since just a very limited number of viewing directions are used and consequently also a potentially very small number of images, the radiation exposure of the person to be examined in the case of X-ray based imaging is significantly reduced compared with fully three-dimensional monitoring conceivable in principle by CT or CBCT. In addition, the computing effort for generating the first reconstruction is kept within reasonable limits because only a few viewing directions have to be taken into account to generate the first reconstruction and not some hundreds or even thousands of viewing directions.

According to at least one embodiment, the hollow organ is a vascular structure, so for example one or more connected vessels, and the implant is a vascular implant.

For example, the hollow organ may be a vascular structure in the brain, so a cerebral vascular structure, for example a cerebral artery, and/or the implant may be a flow diverting implant for taking blood flow in the hollow organ past an aneurysm, for example an aneurysm sac, of the hollow organ. Alternatively, an implant may be what is known as an aneurysm coil, an intra-aneurysmal flow diverter or another neuro stent. It may also be a stent for application in a bile duct, a renal artery stent, and so on.

According to at least one embodiment, the implant is a flow diverting implant for taking blood flow in a hollow organ past an aneurysm of the hollow organ, so for example a flow diverter, and the hollow organ is for example a blood vessel in the brain.

In such embodiments, the solution is advantageous for various reasons. In contrast with, for example, cardiovascular fields of use in which stents or other vascular implants are used, the implants that are employed as flow diverters in the cerebral usage context are structurally far more delicate. One reason for this is that the radial forces that a heart stent must exert on the surrounding blood vessel in order to be able to maintain the blood flow through the blood vessel are many times higher than the radial forces that must act outwards on the cerebral blood vessel from a flow diverter, especially since the issue here is not to open a stenosis or the like but, on the contrary, to prevent the blood flow from the main artery into the aneurysm. Another reason is that the radial opposing forces applied by cerebral arteries are usually many times lower than the opposing forces exerted by blood vessels in the heart or coronary vessels or the like. Hence, flow diverters may have a far more delicate design, but as a consequence this means that in fluoroscopy, for instance X-ray fluoroscopy, they are far more difficult for the person performing the treatment to identify in the underlying two-dimensional images. It is therefore particularly advantageous here to provide the three-dimensional reconstruction for determining the position.

According to at least one embodiment, the image data consists of one or more two-dimensional images, which each represent the implant in the first state inside the hollow organ, and correspond to at most five different viewing directions, for example two or three different viewing directions.

For each viewing direction of the at most five different viewing directions, i.e. a number of one to five viewing directions, the image data thus includes at least one two-dimensional image, although depending on the embodiment, for one or more or all of the given viewing directions, the image data may contain two or more corresponding images of the image data. It is also not absolutely necessary for the number of images in the image data to be the same for different viewing directions.

In such embodiments, the first reconstruction may also reflect to a very good approximation the shape and/or location of the implant, while the computing effort in taking into account at most five, for example two or three, different viewing directions is still very low. In the case of X-ray based imaging for generating the two-dimensional images, the inherent lower X-ray dose resulting from the small number of viewing directions is also advantageous.

According to at least one embodiment, the geometric and/or mechanical model of the implant includes a length and/or diameter of the implant.

The length and/or diameter of the implant may be for example a final length or a final diameter respectively after the implant has fully deployed. It is also possible that the geometric and/or mechanical model of the implant includes different lengths and/or different diameters, if applicable also diameters specified along a longitudinal direction of the implant at different longitudinal positions, for different states, for example deployment states, of the implant. This may further reduce the solution space eligible for generating the three-dimensional reconstruction on the basis of the image data, and therefore may further reduce the computing effort for generating the first reconstruction.

Alternatively or additionally, the geometric and/or mechanical model may also include a radial force or a plurality of radial forces for the final state, initial state, or other states of the implant that lie between the final and initial state. For example, the radial forces may correspond to maximum forces acting outwards in a radial direction that the implant may exert on the surrounding tissue, for example the hollow organ.

In such embodiments, the solution space for generating the first reconstruction may be further reduced with the advantages described above.

According to at least one embodiment, the first reconstruction is generated on the basis of the image data and a pre-intervention representation, for example a three-dimensional pre-intervention representation of the hollow organ.

The pre-intervention representation of the hollow organ may be generated, for example, on the basis of pre-intervention image data, for instance CT image data or CBCT image data, acquired before the intervention, i.e. before the insertion of the implant into the hollow organ. The pre-intervention representation of the hollow organ indicates for example the topology and the geometric properties of the hollow organ. Once the implant is located as intended in the hollow organ, the solution space for generating the first reconstruction may be restricted further by taking into account the pre-intervention representation of the hollow organ, with the advantages described above.

In order to achieve an even more precise or targeted restriction of the solution space, the pre-intervention representation of the hollow organ may also be generated from an initial or undeformed representation of the hollow organ, in order to take into account a deformation of the hollow organ caused by the introduction of the implant or, if applicable, suitable instruments for introducing the implant.

Registration of the two or more first images with respect to each other may be provided for the averaging of the two or more first images, so that the two or more first images are initially registered, or in other words aligned, with respect to one another, and only then is the averaging carried out on the basis of the mutually aligned or registered first images. This has the advantage, for example, that minimal movements of the implant between the different points in time at which the two or more first images were acquired may be corrected, allowing the outline of the implant to be ascertained or extracted more accurately. This is advantageous for example when the implant is expected to move in the specific usage.

In other cases of use, for example for uses in the brain, it may be possible that without corresponding alignment or registration of the first images with one another, averaging of the first images is possible without producing a sub-optimum result from motion artifacts, because for example movements resulting from cardiac movement or movements of the implant arising from respiratory movement or peristaltic movement of the patient to be examined are practically negligible in such uses.

In embodiments, the two or more two-dimensional first images consist of three to five first images.

In such embodiments, the computing effort for the determination is very low and the noise-suppressing effect of the averaging is already significant.

According to at least one embodiment, the image data includes two or more two-dimensional second images, each of which represents the implant in the first state according to a second viewing direction, that differs from the first viewing direction, at different points in time. An averaged second image is generated on the basis of the two or more second images. The first reconstruction is generated on the basis of the averaged second image, for example on the basis of the averaged first image and the averaged second image.

Analogous embodiments also follow for each of all the further of the at most ten viewing directions.

In other words, for each of the at most ten viewing directions, a corresponding averaged image may be generated on the basis of two or more images in the image data that correspond to the associated viewing direction at different points in time, and the first reconstruction is generated on the basis of the averaged images.

According to at least one embodiment, for a marker indicating the position of the implant, a marker position is ascertained in each of the two or more first images. The marker may be located on the implant. The marker may also be located, however, on an auxiliary instrument for inserting the implant into the hollow organ. The two or more first images are aligned, i.e. for example registered, with respect to one another according to the marker positions. The averaged first image is generated on the basis of the aligned two or more first images.

For example, the two or more aligned first images are averaged in order to generate the averaged first image.

The marker position of the marker may be given for example in the image domain of the two or more first images. The auxiliary instrument may be, for example, a catheter or a guide wire for introducing the implant into the vessel.

Thus by the markers, a possible movement of the implant or of the auxiliary instrument made between the individual acquisitions may be identified and corrected by the alignment of the first images with one another. For example, the first images may be aligned with respect to one another in such a way that the marker positions overlie one another in the aligned images.

In further embodiments, corresponding marker positions are ascertained for two or more markers of the implant or auxiliary instrument in each of the two or more first images. The two or more first images are aligned with respect to one another according to the marker positions. The averaged first image is generated on the basis of the aligned two or more first images.

In such embodiments, it is possible to correct not only a translational displacement of the marker positions of an individual marker in the various first images but also a rotation of the implant or the auxiliary instrument over the two or more first images. An even more accurate representation of the implant in the averaged first image may hence be achieved.

Corresponding embodiments are also possible with regard to the second images, that correspond to the second viewing direction, and for all the averaging procedures of the respective images from different viewing directions.

According to at least one embodiment, the two or more first images correspond to images of an image sequence, for example successive images of the image sequence. The image sequence has an image acquisition rate lying in the region of three images per second to fifteen images per second.

Given the considerably longer time scale over which the deployment of the implant proceeds, it may thus be ensured with high reliability that all the first images, that represent the implant in the first state with the first viewing direction at different points in time, also actually represent the same first state of the implant, or in other words differences are negligible.

Analogous embodiments also follow for the two or more second images and/or for images from further viewing directions, that are averaged accordingly.

According to at least one embodiment, an outline of the implant is ascertained by segmenting the averaged first image, and the first reconstruction is generated on the basis of the outline of the implant.

The three-dimensional first reconstruction may thereby be generated with high reliability.

Analogous embodiments also follow for the two or more second images and/or for images from further viewing directions, that are averaged accordingly.

According to at least one embodiment, further image data is obtained consisting of one or more two-dimensional further images, that each represent the implant in a second state inside the hollow organ from at most ten different viewing directions. A three-dimensional second reconstruction of the implant in the second state is generated on the basis of the further image data. The further image data includes two to ten two-dimensional further images having different viewing directions with respect to the implant, and/or the second reconstruction is generated on the basis of the further image data and the geometric and/or mechanical model of the implant. A representation of the second reconstruction is displayed on the display device.

For example, the second state exists after the implant was in the first state. The representation of the obtained reconstruction may thus be understood, for example, as updating the displayed representation from the first reconstruction to the second reconstruction. For example, corresponding image data may also be obtained analogously for further states of the implant, and corresponding three-dimensional reconstructions may be generated and displayed as described.

Thus effectively a real-time representation of the reconstruction of the implant may be displayed. This may provide even faster assistance to the person performing the treatment in assessing the position of the implant in the hollow organ.

According to at least one embodiment, the representation of the first reconstruction contains a superposition of the first reconstruction with a reference representation of the hollow organ, and/or the representation of the second reconstruction includes a superposition of the second reconstruction with the reference representation of the hollow organ.

The reference representation of the hollow organ may be a representation based on a pre-operative three-dimensional dataset, for example a CT dataset or CBCT dataset. If applicable, the reference representation may also be deformed, for instance in order to account for a deformation caused by introducing the implant or the auxiliary instrument into the hollow organ.

Since the representation of the first reconstruction or of the second reconstruction thus also includes the reference representation, the person performing the treatment may interpret better the three-dimensional location of the implant and the shape of the implant without needing to have recourse to a further display of the reference representation or the like.

According to a further aspect, a data processing system is defined, that is configured to perform a computer-implemented method as described herein.

In the present disclosure, the terms “data processing system” and “at least one data processing device” may be used interchangeably. A data processing device may be understood to mean for example a data processing device that contains a processing circuit. Thus the data processing device may process for example data for performing computing operations. These include, if applicable, also operations for performing indexed accesses to a data structure, for instance to a look-up table (LUT), and also a data processing process implemented in hardware.

The data processing device may contain for example one or more computers, one or more microcontrollers, and/or one or more integrated circuits, for example one or more application-specific integrated circuits (ASIC), one or more field-programmable gate arrays (FPGA), and/or one or more systems on a chip (SoC). The data processing device may also contain one or more processors, for example one or more microprocessors, one or more central processing units (CPU), one or more graphics processing units (GPU), and/or one or more signal processors, for example one or more digital signal processors (DSP). The data processing device may also contain a physical or virtual interconnection of computers or other of the aforementioned units.

In various embodiments, the data processing device contains one or more hardware and/or software interfaces and/or one or more memory units.

A memory unit may be configured as a volatile data storage medium, for example as a dynamic random access memory (DRAM) or a static random access memory (SRAM), or as a non-volatile data storage medium, for example as a read-only memory (ROM), as a programmable read-only memory (PROM), as an erasable programmable read-only memory (EPROM), as an electrically erasable programmable read-only memory (EEPROM), as a flash memory or flash EEPROM, as a ferroelectric random access memory (FRAM), as a magnetoresistive random access memory (MRAM), or as a phase-change random access memory (PCRAM).

According to a further aspect, an imaging system is defined, that has a data processing system and an imaging modality. The imaging modality is configured to generate the image data.

For example, the imaging modality may be an X-ray imaging modality, for instance an X-ray angiography system, a C-arm X-ray system, and so on.

According to a further aspect, a computer program is defined, that has commands that, on execution by a data processing system, cause the data processing system to perform the computer-implemented method described herein.

For example, the commands may exist as program code. The program code may be provided, for example, as binary code or assembler and/or as source code of a programming language, for instance C, and/or as program script, for instance Python.

According to a further aspect, a further computer program is defined, that has further commands that, on execution by an imaging system, for example by the data processing system of the imaging system, cause the data processing system to perform a computer-implemented method, and cause the imaging modality to generate the image data.

For example, the further commands may exist as program code. The program code may be provided, for example, as binary code or assembler and/or as source code of a programming language, for instance C, and/or as program script, for instance Python.

According to a further aspect, a computer-readable storage medium is defined, for example a physical and/or non-volatile computer-readable storage medium, that stores a computer program and/or a further computer program.

The computer program, the further computer program and the computer-readable storage medium are each computer program products containing the commands and/or the further commands.

Further features and combinations of features of appear in the figures and the description of the figures and in the claims. For example, further embodiments need not necessarily contain all the features of one of the claims. Further embodiments may have features and combinations of features that are not mentioned in the claims.

Embodiments are described in greater detail below with reference to specific embodiments and associated schematic drawings. In the figures, identical or functionally equivalent elements may be denoted by the same reference signs. The description of identical or functionally equivalent elements is not necessarily repeated when referring to different figures.

### BRIEF DESCRIPTION OF THE FIGURES

FIG. 1 depicts a schematic representation of an embodiment of an imaging system.

FIG. 2 depicts a schematic flow diagram of an embodiment of a computer-implemented method for determining the position of an implant in a hollow organ.

FIG. 3 depicts a schematic representation of a two-dimensional image, that depicts an implant and an auxiliary instrument for inserting the implant into a hollow organ according to an embodiment.

FIG. 4 depicts a schematic flow diagram of a further embodiment of a computer-implemented method for determining the position of an implant in a hollow organ.

FIG. 5 depicts a schematic flow diagram of a further embodiment of a computer-implemented method for determining the position of an implant in a hollow organ.

FIG. 6 depicts a schematic representation of two-dimensional images, that show an implant and an auxiliary instrument for inserting the implant into a hollow organ.

### DETAILED DESCRIPTION

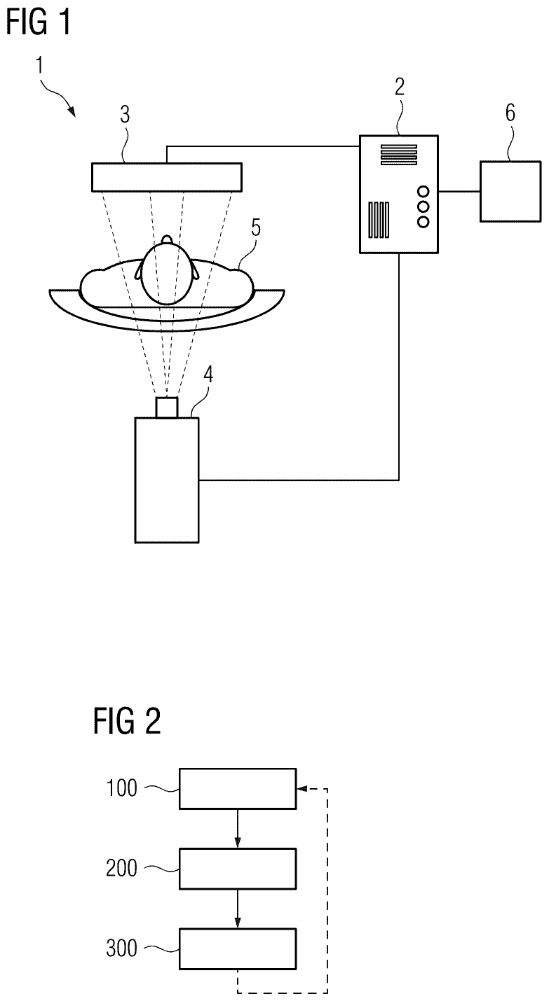

FIG. 1 depicts a schematic representation of an embodiment of an imaging system 1. The imaging system 1 may be used to determine the position of an implant 9, for example a flow diverter, in a hollow organ, for instance a cerebral artery, of a patient 5.

The imaging system 1 includes an imaging modality, for instance an X-ray imaging modality 3, 4. The imaging modality is configured to generate image data 7, 7a, 7b, 7c, 8, 8a, 8b, 8c consisting of one or more two-dimensional images 7, 7a, 7b, 7c, 8, 8a, 8b, 8c, that each represent the implant 9 in a first state inside the hollow organ and correspond to at most ten different viewing directions.

The imaging system 1 includes a data processing system 2 including a display device 6. The data processing system 2 is configured to perform a computer-implemented method for determining the position of an implant 9, for example a flow diverter, in a hollow organ, for example a cerebral artery, for example on the basis of the image data 7, 7a, 7b, 7c, 8, 8a, 8b, 8c.

If the imaging modality is configured as an X-ray imaging modality 3, 4, then it includes a source unit 3 including an X-ray source, a detector unit 4 including an X-ray detector, and a control system, that is configured to control the X-ray source and the X-ray detector in order to generate the two-dimensional images 7, 7a, 7b, 7c, 8, 8a, 8b, 8c.

FIG. 2 depicts a schematic flow diagram of an embodiment of a computer-implemented method for determining the position of an implant 9 in a hollow organ.

In step 100, the image data 7 is obtained, for example from the imaging modality. In step 200, a three-dimensional first reconstruction of the implant 9 in the first state is generated on the basis of the image data 7. The image data 7 includes two to ten two-dimensional images 7 each including different viewing directions with respect to the implant 9, and/or the first reconstruction is generated on the basis of the image data 7 and a predefined geometric and/or mechanical model of the implant 9. In step 300, a representation of the first reconstruction is displayed on the display device 6.

The steps 100 to 300 may be repeated multiple times, and in each repetition another state of the implant may exist and be represented by the image data 7. It is thereby possible to achieve a dynamic real-time representation of the implant 9.

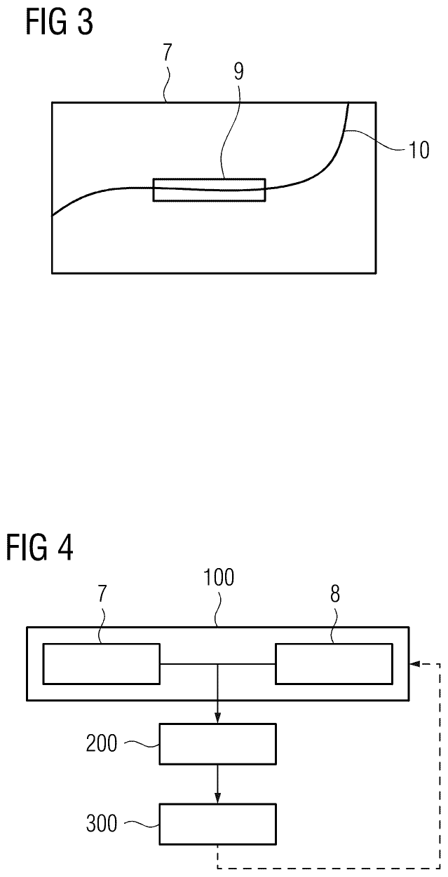

FIG. 3 depicts schematically a two-dimensional image 7, that depicts the implant 9, for example a stent or flow diverter, and an auxiliary instrument 10, for example a guide wire, for introducing the implant 9 into the hollow organ.

FIG. 4 depicts a schematic flow diagram of an embodiment of a computer-implemented method that is based on the embodiment of FIG. 2.

In the embodiment of FIG. 4, the image data 7, 8 contains a first two-dimensional image 7, that depicts the implant in a first viewing direction, and a second two-dimensional image 8, that depicts the implant in a second viewing direction. For this purpose, the imaging modality may be in the form of a biplane X-ray imaging modality 3, 4, for example. The first reconstruction may be generated with greater accuracy by taking into account two different viewing directions.

FIG. 5 depicts a schematic flow diagram of an embodiment of a computer-implemented method that is based on the embodiment of FIG. 4.

In the embodiment of FIG. 4, the image data 7a, 7b, 7c, 8a, 8b, 8c contains a plurality of, for instance three, two-dimensional first images 7a, 7b, 7c, that show the implant 9 at different points in time each in the first viewing direction, and a plurality of, for instance three, two-dimensional second images 8a, 8b, 8c, that show the implant at different points in time each in the second viewing direction.

In step 200, an averaged first image is generated on the basis of the first images 7a, 7b, 7c, and an averaged second image is generated on the basis of the second images 8a, 8b, 8c. The first reconstruction is generated on the basis of the averaged first image and the averaged second image.

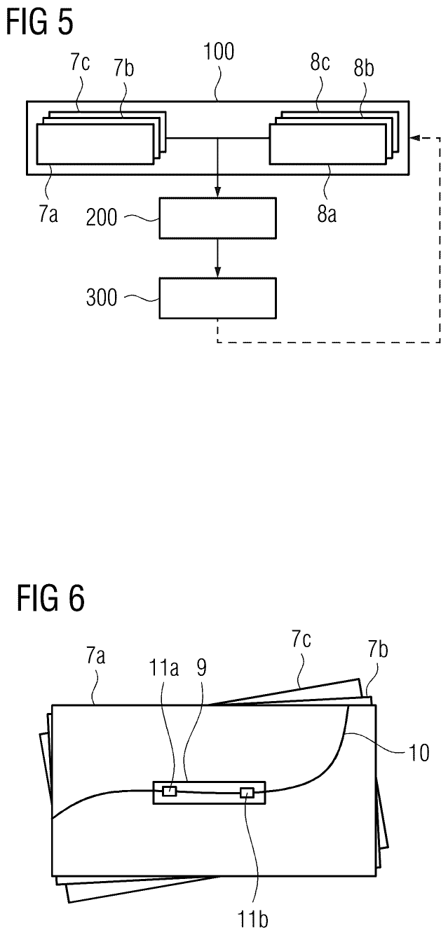

One or more markers 11a, 11b, for instance two markers 11a, 11b, that are X-ray opaque, for example, may be provided on the implant 9 or the auxiliary instrument 10. This is shown schematically in FIG. 6. For each of the markers 11a, 11b, a marker position is ascertained in each of the first images 7a, 7b, 7c, for example. The first images 7a, 7b, 7c are aligned with respect to one another depending on the marker positions, for example so that the respective markers in different aligned first images 7a, 7b, 7c overlie one another. The averaged first image is generated on the basis of the aligned first images 7a, 7b, 7c, and the averaged second image is generated on the basis of the aligned second images 8a, 8b, 8c.

Visual accentuation or highlighting of the implant 9 and noise suppression by averaging are used in various embodiments as described for example with reference to FIG. 4. For this purpose, an improved, for example sharpened, version of the implant 9 may be calculated by averaging at regular intervals, for example on the basis of a sliding time window, that covers a series of successively acquired two-dimensional images 7, 7a, 7b, 7c, 8, 8a, 8b, 8c. In some embodiments, the images 7, 7a, 7b, 7c, 8, 8a, 8b, 8c may first be registered by the markers 11a, 11b, and then averaged according to the current position of the sliding time window.

Since movements in the imaging of the brain usually do not constitute a significant problem, the averaged images may also be generated without registration, if applicable. This also means that in many cases it is sufficient to average a small number of successive images 7, 7a, 7b, 7c, 8, 8a, 8b, 8c within the current position of the sliding time window.

In some embodiments, a method for symbolic reconstruction is employed in order to generate the first reconstruction of the implant 9. In this case, a severely limited number of viewing directions is sufficient. The symbolic reconstruction is well suited to objects that have a sufficiently simple geometric structure, so also for implants 9, for example flow diverters.

In some embodiments, the markers 11a, 11b are tracked for two different viewing directions. This may be performed, for example, by an algorithm based on a trained machine learning model, for example a trained artificial neural network. Two averaged images are generated for the two viewing directions. The implant is segmented in each of the averaged images, for example using a known analytical or data-controlled approach. Then a symbolic reconstruction of the current status of the implant 9 is performed, for example in real time, and an updated 3D representation of the reconstruction result is displayed.

In some embodiments, instead of visualizing just the implant 9 by its three-dimensional reconstruction, the reconstruction may be merged with pre-intervention planning and displayed jointly in real time in order to allow an even better assessment of whether the expected deployment result is sufficiently close to the planned result. If the current status differs significantly from the result of the pre-intervention planning, the implant 9 may be retracted, for example, and the insertion restarted. For example, another implant 9 may also be selected for insertion. This may be the case, for example, if the result of the pre-intervention planning is based on inaccurate data on the vessel geometry or if the geometric deformation of the vessel, for instance by the implant 9, has been estimated inaccurately.

In some embodiments, for example in the case of geometric ambiguities, a pre-intervention 3D angiographic image may be used in order to restrict by an algorithm the solution space during the symbolic reconstruction. The pre-intervention 3D angiographic image may be updated with the aid of suitable deformation correction techniques, such as those known for example from the field of EVAR guidance (endovascular aneurysm repair guidance).

In some embodiments, a geometric and/or mechanical model of the implant 9 may be used in order to restrict the solution space during the symbolic reconstruction steps.

Further advantages include a greater accuracy in placement of the implant 9 and a potential reduction in the number of implants 9 to be inserted until the desired result is achieved, in order to reduce devices. Both improve the treatment results and lower the procedure costs, for example costs of materials.

It is to be understood that the elements and features recited in the appended claims may be combined in different ways to produce new claims that likewise fall within the scope of the present embodiments. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present embodiments have been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer-implemented method for determining the position of an implant in a hollow organ, the method comprising:
obtaining image data comprising two-dimensional images that each represent the implant in a first state inside the hollow organ from a total of at most ten different viewing directions, wherein the image data includes two to ten two-dimensional images each having different viewing directions with respect to the implant, wherein the image data includes two or more two-dimensional first images, each of which represents the implant in the first state according to a first viewing direction at different points in time;
generating a three-dimensional first reconstruction of the implant in the first state based on the image data;
generating at least one averaged first image based on the two or more first images;
generating the first reconstruction based on the at least one averaged first image; and
diplaying a representation of the first reconstruction on a display device.

2. The computer-implemented method of claim 1, wherein the first reconstruction is generated on the basis of the image data and a predefined geometric and/or mechanical model of the implant.

3. The computer-implemented method of claim 1, wherein the hollow organ is a vascular structure and the implant is a vascular implant.

4. The computer-implemented method of claim 3, wherein the vascular implant is a flow diverting implant for taking blood flow in the hollow organ past an aneurysm of the hollow organ.

5. The computer-implemented method of claim 1, wherein the image data consists of one or more two-dimensional images, that each represent the implant in the first state inside the hollow organ and correspond to at most five different viewing directions.

6. The computer-implemented method of claim 1, wherein the geometric and/or mechanical model of the implant includes a length and/or diameter of the implant.

7. The computer-implemented method of claim 1, wherein the first reconstruction is generated on the basis of the image data and a pre-intervention representation of the hollow organ.

8. The computer-implemented method of claim 1, further comprising:
ascertaining, for a marker indicating the position of the implant, a marker position in each of the two or more first images;
aligning the two or more first images with respect to one another according to the marker positions; and
wherein the the averaged first image is generated on the basis of the aligned two or more first images.

9. The computer-implemented method of claim 1, wherein the two or more first images correspond to images of an image sequence that has an image acquisition rate lying of three images per second to 15 images per second.

10. The computer-implemented method of claim 1, wherein an outline of the implant is ascertained by segmenting the averaged first image, and the first reconstruction is generated on the basis of the outline of the implant.

11. The computer-implemented method of claim 1, further comprising:
obtaining further image data consisting of one or more two-dimensional further images, that each represent the implant in a second state inside the hollow organ from at most ten different viewing directions;
generating a three-dimensional second reconstruction of the implant in the second state on the basis of the further image data, wherein the further image data includes two to ten two-dimensional further images having different viewing directions with respect to the implant, and/or the second reconstruction is generated based on the further image data and the geometric and/or mechanical model of the implant; and
displaying a representation of the second reconstruction on the display device.

12. The computer-implemented method of claim 1, wherein the representation of the first reconstruction includes a superposition of the first reconstruction with a reference representation of the hollow organ.

13. An imaging system comprising:
an imaging modality configured to acquired image data comprising two-dimensional images that each represent the implant in a first state inside the hollow organ from a total of at most ten different viewing directions, wherein the image data includes two to ten two-dimensional images each having different viewing directions with respect to the implant, wherein the image data includes two or more two-dimensional first images, each of which represents the implant in the first state according to a first viewing direction at different points in time; and
a data processing system configured to:
generate a three-dimensional first reconstruction of the implant in the first state based on the image data;
generate at least one averaged first image based on the two or more first images;
generate the first reconstruction based on the at least one averaged first image; and
diplay a representation of the first reconstruction on a display device.

14. A non-transitory computer implemented storage medium, including machine-readable instructions stored therein for determining the position of an implant in a hollow organ, the machine-readable instructions when executed by at least one processor, cause the processor to:
obtain image data comprising two-dimensional images that each represent the implant in a first state inside the hollow organ from a total of at most ten different viewing directions, wherein the image data includes two to ten two-dimensional images each having different viewing directions with respect to the implant, wherein the image data includes two or more two-dimensional first images, each of which represents the implant in the first state according to a first viewing direction at different points in time;
generate a three-dimensional first reconstruction of the implant in the first state based on the image data;
generate at least one averaged first image based on the two or more first images;
generate the first reconstruction based on the at least one averaged first image; and
diplaya representation of the first reconstruction.
